(19) United States
(12) Patent Application Publication
(10) Pub. No.: US 2023/0414225 A1
(43) Pub. Date: Dec. 28, 2023

(54) ENDOSCOPIC TREATMENT TOOL AND CLIP DEVICE

(71) Applicant: OLYMPUS MEDICAL SYSTEMS CORP., Tokyo (JP)

(72) Inventors: Kensuke Uesaka, Hino-shi (JP); Kazuki Akiyoshi, Hirosaki-shi (JP); Toshinori Tamura, Hirosaki-shi (JP); Satoshi Aoki, Hirosaki-shi (JP); Tsutomu Okada, Tachikawa-shi (JP)

(21) Appl. No.: 18/462,880

(22) Filed: Sep. 7, 2023

Related U.S. Application Data
(63) Continuation of application No. PCT/JP21/09575, filed on Mar. 10, 2021.

(51) Int. Cl. A61B 17/128; A61B 17/122

(52) U.S. Cl. CPC A61B 17/1285; A61B 17/122; A61B 2017/12004

(57) ABSTRACT

The endoscopic treatment tool includes a clip having a plurality of arms freely open and close at a distal side of the arm, a holding tube into which at least a portion on a proximal side is inserted, and a connecting member connecting with the clip. The holding tube includes a tightening member provided in an inner region of the holding tube, and a protrusion-retraction wing configured to protrude and retract with respect to an outer side, the tightening member is provided on a proximal side from the protrusion-retraction wing, the clip has an engagement portion on an accommodation region accommodated in the inner region, and a movement of the clip toward a distal side with respect to the holding tube is restricted by engaging the engagement portion moved to the proximal side with the tightening member.

The present disclosure relates to an endoscopic treatment instrument and a clip device. This application is a continuation application based on International Patent Application No. PCT/JP2021/009575 filed on Mar. 10, 2021, and the content of the PCT international application is incorporated herein by reference.

### BACKGROUND

In endoscopic treatment, an endoscopic treatment tool such as a clip unit capable of hemostasis or the like by ligating a resected portion after treatment is used. The clip unit includes a clip that clamps the resected portion, and a holding tube that accommodates the clip and locks the clip in a closed state.

In a case that the clip has a self-expanding force such that the clip is energized to be in an open state, before the clip clamping the resected portion or the like is accommodated in the holding tube and locked in the closed state, the operator can return the clip in the open state and can re-grasp the resected portion or the like.

The clip treatment tool having a clip and a tightening member is described in Patent Document 1. The clip treatment tool described in Patent Document 1 can maintain a tightening of the clip by contacting the convex portion formed on the clip with the ring-shaped tightening member. Further, the clip treatment tool described in Japanese Patent Application Publication No. 2010-029629 can release the tightening of the clip by making the projection formed on the clip not to contact with the ring-shaped tightening member.

In view of the above circumstances, it is an object of the present disclosure to provide an endoscopic treatment tool and a clip device such as a clip unit or the like, which are capable of securely locking the clip having a self-expanding force and re-glaspable into the closed state by the holding tube.

### SUMMARY

In order to solve the above problems, the present disclosure proposes the following means.

A treatment instrument for an endoscope according to a first aspect of the present disclosure includes a clip having a plurality of arms freely open and close at a distal side of the arm, and a holding tube into which at least a portion on a proximal side is inserted. The holding tube includes a tightening member provided in an inner region of the holding tube, and a protrusion-retraction wing configured to protrude and retract with respect to an outside, the tightening member is provided on a proximal side from the protrusion-retraction wing, the clip has an engagement portion on an accommodation region accommodated in the inner region, and a movement of the clip toward a distal side with respect to the holding tube is restricted by engaging the engagement portion moved to the proximal side with the tightening member.

A clip device according to a second aspect of the present disclosure includes the above-described endoscopic treatment instrument and a clip introducer.

### BRIEF DESCRIPTION OF DRAWINGS

FIG. 1 is a figure showing a clip introducer of the clip device according to the embodiment.

FIG. 2 is a cross-sectional view of a wire of the same clip introducer.

FIG. 3 is a cross-sectional view of an operating section main body of an operating section of the clip introducer.

FIG. 4 is a perspective view of the clip unit of the clip device.

FIG. 5 is a perspective view of the same clip unit in which a holding tube illustrates transparent.

FIG. 6 is a perspective view of the holding tube of the same clip unit.

FIG. 7 is a cross-sectional view of the same holding tube along the central axis.

FIG. 8 is cross-sectional view showing the same clip unit into which the sheath of the same clip introducer is loaded.

FIG. 9 is a figure showing the same clip unit introduced into the human body.

FIG. 10 is a figure showing the same clip unit in which a pair of arms is closed.

FIG. 11 is a figure showing the same clip unit in which the clip is locked.

FIG. 12 is a figure showing the same clip unit from which the clip is separated.

FIG. 13 is a figure showing the same clip unit after braking.

FIG. 14 is a perspective cross-sectional view showing a different incorporation mode of a tightening member.

FIG. 15 is a perspective view showing the holding tube of the clip unit according to the second embodiment.

FIG. 16 is a cross-sectional view of the same holding tube along the central axis.

FIG. 17 is a cross-sectional view of the same holding tube along the central axis.

FIG. 18 is a figure for explaining an attaching method of the tightening member of the same clip unit to a holding member main body.

FIG. 19 is a cross-sectional view showing a modified example of the same tightening member.

FIG. 20 is a cross-sectional view showing another modified example of the same tightening member.

FIG. 21 is a cross-sectional view showing a modified example of the same holding tube.

FIG. 22 is a cross-sectional view showing the same modified example of the same holding tube.

FIG. 23 is a cross-sectional view showing another modified example of the same tightening member.

FIG. 24 is a cross-sectional view showing another modified example of the same tightening member.

FIG. 25 is a cross-sectional view of the same modified example.

FIG. 26 is a figure showing another attachment aspect of the same modified example of the tightening member.

FIG. 27 is a cross-sectional view of another modified example of the tightening member.

FIG. 28 is a cross-sectional view of another modified example of the holding tube main body.

FIG. 29 is a cross-sectional view of the same modified example.

FIG. 30 is a cross-sectional view showing a modified example of the same holding tube.

FIG. 31 is a perspective view showing the holding tube of the clip unit according to the third embodiment.

FIG. 32 is a side view of the same clip unit.

FIG. 33 is a perspective view of a pair of arms of the same clip unit.

FIG. 34 is an enlarged figure of a region R1 in FIG. 33.

FIG. 35 is a figure showing the same pair of arms partially accommodated in the holding tube of the same clip unit.

FIG. 36 is a side view of a base end portion of the same pair of arms.

FIG. 37 is a cross-sectional view of the same clip unit introduced into a sheath of the clip introducer.

FIG. 38 is a cross-sectional view of a protrusion-retraction wing of the same holding tube.

FIG. 39 is a cross-sectional view of a modified example of the same protrusion-retraction wing.

FIG. 40 is a side view of the same clip unit protruded from the sheath toward the tip side.

FIG. 41 is a figure showing a modified example of a holding pipe of the same holding tube.

FIG. 42 is a figure showing another modified example of a holding pipe of the same holding tube.

FIG. 43 is a figure showing another modified example of a holding pipe of the same holding tube.

### DETAILED DESCRIPTION OF EXEMPLARY EMBODIMENTS

### First Embodiment

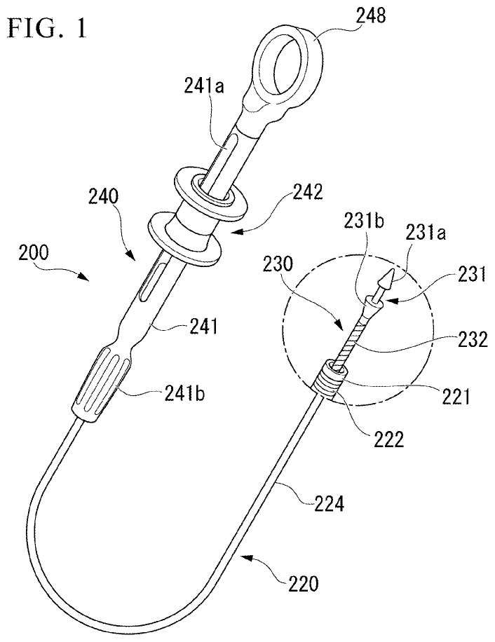

A first embodiment of the present disclosure will be described with reference to FIGS. 1 to FIG. 14. A clip device 100 according to this embodiment includes a clip unit (endoscopic treatment tool) 1 and a clip introducer 200 for operating the clip unit 1. The clip unit 1 is used by loading the clip unit 1 into the clip introducer 200.

### [Clip Introducer 200]

FIG. 1 is a figure showing the clip introducer 200. The clip introducer 200 includes a sheath 220, an operating wire 230, and an operating section 240. The clip introducer 200 is inserted through, for example, a treatment instrument insertion channel of an endoscope and used in combination with the endoscope. Therefore, the sheath 220 is formed sufficiently longer than the length of the treatment instrument insertion channel of the endoscope. The sheath 220 is flexible and is capable of curving in accordance with the curvature of the insertion portion of the endoscope.

The sheath 220 includes a distal tip 221, a distal end side coil 222, and a proximal coil 224. The sheath 220 is formed in an elongated tubular shape as a whole. The distal end side coil 222 is arranged on the distal portion side of the sheath 220. The distal tip 221 is arranged at a tip end portion of the distal end side coil 222.

The operation wire (power transmitter) 230 includes an arrowhead hook section (connection portion) 231 connected to the clip unit 1 and a wire 232 for operating the arrowhead hook section 231, as shown in FIG. 1.

The arrowhead hook portion 231 includes an engagement portion 231 which has a substantially conical shape and engages with the clip unit 1, and a wire connection portion 231b provided at a proximal end of the engagement portion 231a. The arrowhead hook portion 231 is made of a metal material such as stainless steel, for example, SF20F and SUS303.

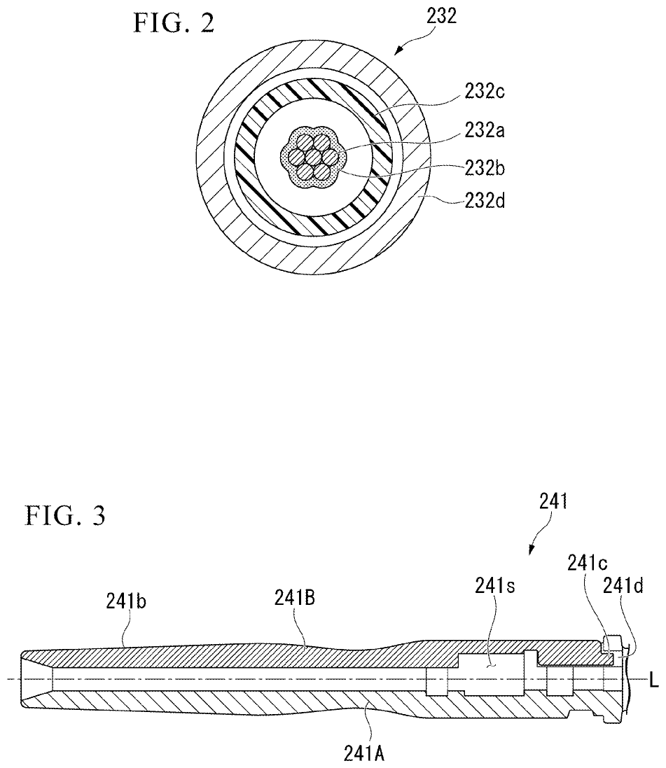

FIG. 2 is a cross-sectional view of wire 232. The wire 232 is inserted through the sheath 220 so as to freely advance and retract. The wire 232 has a general-purpose medical wire 232a that is not coating treated, a tube 232c, and a coil sheath 232d. A silicone oil 232b is applied as a lubricant to an outer surface of the medical wire 232a. The medical wire 232a is passed through the inside of the tube 232c. The tube 232c is inserted through the coil sheath 232d. A tip end portion of the wire 232 is fixed to a proximal end of the wire connection portion 231b by, for example, welding or caulking.

The operating section 240 includes an operating section main body 241, a slider 242, and a thumb ring 248, as shown in FIG. 1. The operating section main body 241 is, for example, injection-molded from a resin material. The operating section main body 241 includes a slit portion 241a, and a rotary grip 241b positioned on the tip end side of the operation portion main body 241. The slit portion 241a supports the slider 242 so that the slider is capable of advancing and retracting along the longitudinal axis L.

The slider 242 is attached to be advanceable and retractable in the longitudinal axis direction of the operating section main body 241, and the proximal end of the wire 232 is attached to the slider 242. As the slider 242 advances and retracts along the operating section main body 241, the wire 232 advances and retracts with respect to the sheath 220, and the arrowhead hook portion 231 advances and retracts.

The thumb ring 248 is attached to the proximal end of the operating section main body 241 so as to be rotatable about the longitudinal axis of the operating section main body 241.

FIG. 3 is a cross-sectional view of the operating section main body 241 of the operating section 240. The operating section main body 241 has an operating section base 241A and an operating section lid 241B. The operating section lid 241B is a lid that closes a groove extending along the longitudinal axis L formed in the operating section base 241A. The operating section base 241A and the operating section lid 241B form an internal space 241s of the operating section main body 241. A convex portion 241c formed on the proximal end side (thumb ring 248 side) of the operating section lid 241B engages with a concave portion 241d formed on the operating section base 241A. Since the convex portion 241c and the concave portion 241d are engaged so as not to move relative to each other in the direction orthogonal to the longitudinal axis L, the operating section lid 241B is less likely to come off from the operating section base 241A when the slider 242 is operated.

### [Clip Unit 1]

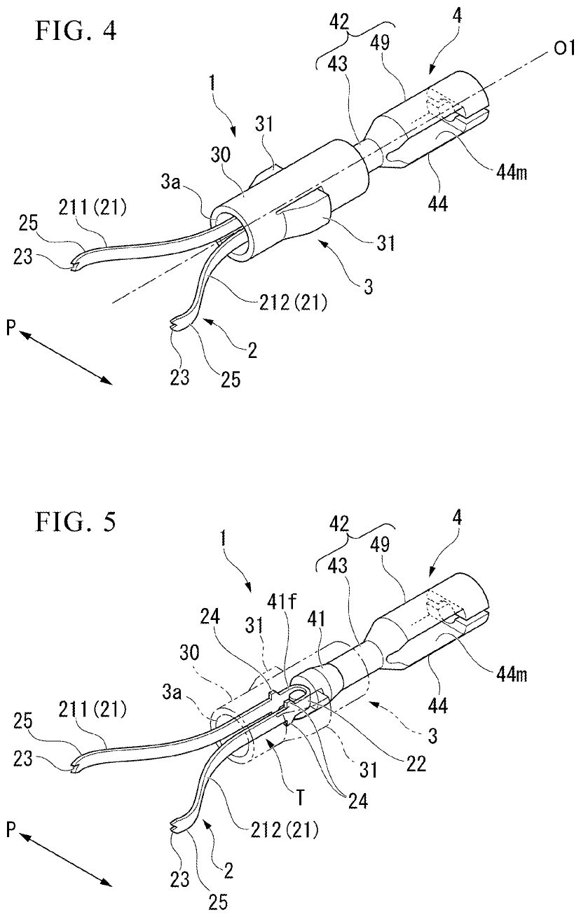

FIG. 4 is a perspective view of the clip unit 1 according to the embodiment. FIG. 5 is a perspective view of the clip unit 1 in which the holding tube 3 is transparently illustrated. The clip unit (treatment instrument for an endoscope) 1 includes a clip 2, a holding tube 3 having substantially cylindrical shape, and a connecting member 4. In the following description, the clip 2 side is defined as the tip side (distal side) of the clip unit, and the connecting member 4 side is defined as the base end side (proximal side) of the clip unit 1.

The clip 2 is formed by bending a plate made of metal such as a plate spring made of stainless steel, cobalt-chromium alloy, or the like, at the center. The clip 2 has a pair of arms 21 that is openenable and closable, and a connecting portion 22 that connects the pair of arms 21.

The pair of arms 21 has a first arm 211 and a second arm 212. The first arm 211 and the second arm 212 are arranged symmetrically with respect to a central axis O1 in the longitudinal direction of the clip unit 1. Tissue grasping portions 23 facing each other are formed at the tip ends of the pair of arms 21, and have curved portions 25 that bulge outward with respect to the central axis O1. The tissue grasping portion 23 is formed by bending the tips of the pair of arms 21 inward.

An engagement portion 24 projecting in a direction perpendicular to the central axis O1 is formed on the base side of the first arm 211 and on the tip side of the second arm 212. The tissue grasping portion 23 side of the engagement portion 24 is formed with an acute-angled slope, and the connecting portion 22 side of the engagement portion 24 is formed with an obtuse-angled slope. The engagement portion 24 is provided in an accommodated region T that is accommodated in an inner area S of the holding tube 3. The accommodated region T is a region that is always accommodated in the inner region S of the holding tube 3 when the pair of arms 21 is opened and closed.

The first arm 211 and the second arm 212 are connected by the connecting portion 22 on the base end side, and are provided so as to be openable and closable toward the tip side. The connecting portion 22 is formed to have a U-shape by being bent and is connected to the connecting member 4. The connecting portion 22 is biased so that the pair of arms 21 is in an open state. Therefore, the pair of arms 21 of the clip 2 has a self-expanding force in the opening/closing direction P.

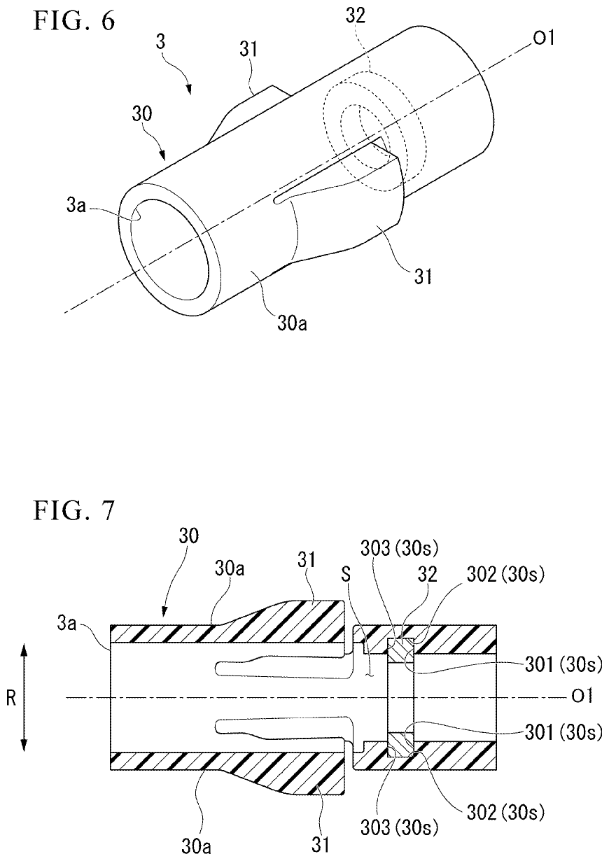

FIG. 6 is a perspective view of the holding tube 3.

The holding tube 3 has a holding tube main body 30 formed in a cylindrical shape, a protrusion-retraction wing 31, and a tightening member 32.

The holding tube main body 30 is made of a material softer than that of the clip 2, such as PPA (polyphthalamide), PA (polyamide), PEEK (polyetheretherketone), LCP (liquid crystal polymer), or other thermoplastic resin having moderate elasticity, is formed by injection molding. The holding tube main body 30 may be made of metal instead of thermoplastic resin.

FIG. 7 is a cross-sectional view of the holding tube 3 along the central axis O1. The protrusion-retraction wings 31 are a pair of protrusions projecting with respect to an outer peripheral surface 30a of the holding tube main body 30. The protrusion-retraction wings 31 are provided on both sides of the center axis O1. The protrusion-retraction wings 31 have a basic posture in which each of the protrusion-retraction wings 31 projects outward in the radial direction R with respect to the outer peripheral surface 30a. The protrusion-retraction wings 31 are in a retracted state in which the protrusion-retraction wings 31 are retracted with respect to the outer peripheral surface 30a by receiving a force directed from the outside toward the inside in the radial direction R. By releasing the above force, the protrusion-retraction wings 31 return from the retracted state to the projected state.

The tightening member 32 is a ring-shaped member provided in an inner region S of the holding tube 3. The tightening member 32 is made of metal. The tightening member 32 may be formed so as to be harder than the holding tube main body 30, and may be formed of thermoplastic resin instead of metal, for example.

The tightening member 32 is arranged such that the central axis of the tightening member 32 coincides with the central axis O1. The tightening member 32 is arranged on the base end side of the protrusion-retraction wing 31. The tightening member 32 is incorporated into the holding tube main body 30 by, for example, insert molding. The base end side of the tightening member 32 is in contact with a support surface 30s formed on the holding tube main body 30. The support surface 30s has a first support surface 301, a second support surface 302, and a third support surface 303.

The first support surface 301 is a surface that supports the tightening member 32 from the base end side, and is a surface that is substantially perpendicular to the central axis O1. As shown in FIG. 7, the surface of the tightening member 32 that contacts with the support surface 301 is preferably substantially perpendicular to the central axis O1.

The second support surface 302 is a surface that supports the tightening member 32 from the outer circumferential side, and is a surface that is substantially parallel to the central axis O1. As shown in FIG. 7, the outer peripheral surface of the tightening member 32 that contacts with the second support surface 302 is preferably substantially parallel to the second support surface 302.

The third support surface 303 is a surface that supports the tightening member 32 from the tip side, and is a surface that is substantially perpendicular to the central axis O1. As shown in FIG. 7, the surface of the tightening member 32 that contacts with the third support surface 303 is preferably substantially perpendicular to the central axis O1.

The connecting member 4 is connected to the connecting portion 22 of the clip 2. The connecting member 4 is connected to the arrowhead hook portion 231 that passes through the sheath 220. That is, the connecting member 4 connects the clip 2 with the arrowhead hook portion 231. The connecting member 4 includes an insertion portion 41 inserted into the inner space of the holding tube 3, and a connecting portion 42 provided at the base end of the insertion portion 41.

The insertion portion 41 has a hook 41f at a tip end portion of the insertion portion 41. The hook 41f is a hook extending in a direction perpendicular to the central axis O1, and is formed in a substantially columnar rod shape. The connecting portion 22 of the clip 2 is hooked on the hook 41f. The hook 41f is broken when the connecting portion 22 is pulled toward the base end side and a breaking force of, for example, 20N (Newton) to 60N is applied to the hook 41f.

The connecting portion 42 is an engagement portion with which the arrowhead hook portion 231 of the clip introducer 200 is engaged (connected). The connecting portion 42 has a connecting portion main body 43, and an elastic arm portion 44.

The elastic arm portion 44 is provided at the base end of the connecting portion main body 43, and is branched into two. The elastic arm portion 44 is elastically deformable with respect to the connecting portion main body 43, and is openable and closable with respect to the connecting portion main body 43. The elastic arm portion 44 is formed with a notch portion 44m for grasping and accommodating the engagement portion 231a of the arrowhead hook portion 231. The notch portion 44m is formed in a shape that closely contacts with an outer peripheral surface of the engagement portion 231a of the arrowhead hook portion 231.

### [Actions and Effects of Clip Unit 1]

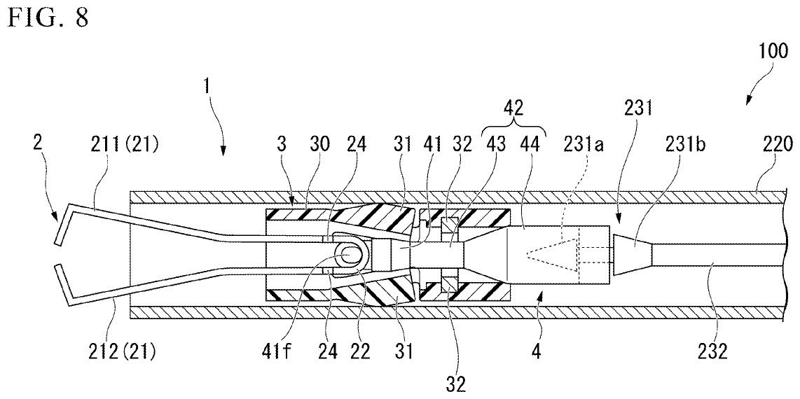

Next, actions and effects of the clip unit 1 will be described with reference to FIGS. 8 to 12.

FIG. 8 is a cross-sectional view showing the clip unit 1 loaded into the sheath 220 of the clip introducer 200. The connecting member 4 of the loaded clip unit 1 is connected with the arrowhead hook portion 231 inserted through the sheath 220. The protrusion-retraction wings 31 are in a retracted state by being pressed by the inner peripheral surface of the sheath 220.

The pair of arms 21 of the loaded clip unit 1 is in a closed state by being pressed by the inner peripheral surface of the sheath 220. The engagement portion 24 is located on the tip end side from the tightening member 32, and the pair of arms 21 is not locked in the closed state.

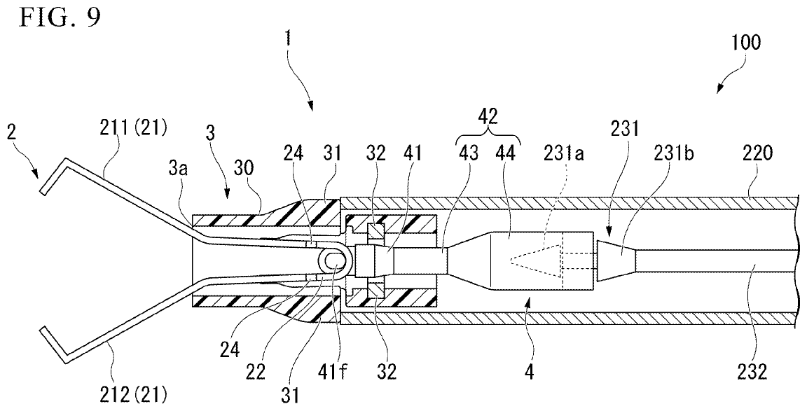

FIG. 9 shows the clip unit 1 introduced into the body.

The user introduces the clip unit 1 loaded into the sheath 220 into the body through the channel of the endoscope. Next, the user advances the arrowhead hook portion 231 by advancing the slider 242 along the operating section main body 241. The user advances the clip unit 1 until the protrusion-retraction wings 31 are exposed from the sheath 220. The protrusion-retraction wings 31 are return from the retracted state to the projected state, which is the basic posture in accordance to the exposure of the protrusion-retraction wings 31 from the sheath 220.

When the tip end sides of the pair of arms 21 is exposing from the sheath 220, while the clip 2 is moving toward the tip end side with respect to the holding tube 3, thereby the clip 2 returns to the open state due to the self-expanding force of the pair of arms 21 as a restoring force. Even when the pair of arms 21 returns to the open state and most protrude from the holding tube 3, the engagement portion 24 is arranged in the inner region S of the holding tube 3.

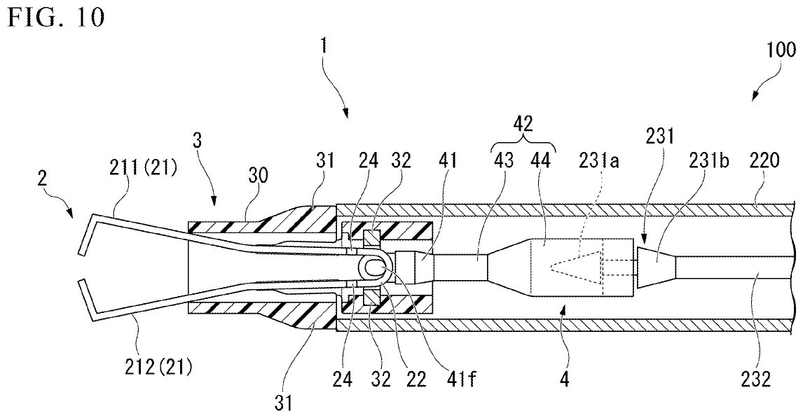

FIG. 10 shows the clip unit 1 when the pair of arms 21 is closed.

The user retracts the arrowhead hook portion 231 by retracting the slider 242 along the operating section main body 241. The connecting member 4 connected to the arrowhead hook portion 231 pulls the clip 2. A pair of arms 21 having a self-expanding force pushes the tip opening 3a of the holding tube toward the base end side by being pulled toward the base end side. Since the protrusion-retraction wings 31 in the protruding state engages with the sheath 220, the protrusion-retraction wings 31 are not drawn into the sheath 220. Therefore, the clip 2 pulled by the connecting member 4 is drawn into the holding tube 3.

The connecting portion 22 of the clip 2 is pulled by the connecting member 4 toward the base end side of the holding tube 3, so that the pair of arms 21 is drawn into the holding tube 3, and the pair of arms 21 gradually closes. When the pulling force of the connecting portion 22 is released in this state, the self-expanding force of the pair of arms 21 is used as a restoring force, and the clip 2 returns to the open state while moving the clip 2 toward the tip end side. The user can return the pair of arms 21 to the open state and re-grasp the tissue.

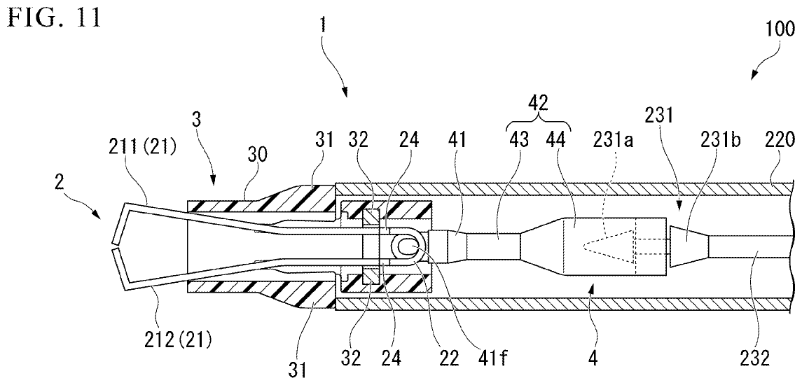

FIG. 11 is the figure showing the clip unit 1 when the clip 2 is locked.

As the connecting portion 22 is further pulled toward the base end side of the holding tube 3, the engagement portion 24 is pulled to the proximal end side from the tightening member 32. Since the connecting portion 22 side of the engagement portion 24 is formed as an obtuse slope, the engagement portion 24 is easily retracted from the tightening member 32 to the base end side. On the other hand, since the tissue grasping portion 23 side of the engagement portion 24 is formed to have an acute slope, when the engagement portion 24 is pulled to the base end side of the tightening member 32, the engagement portion 24 and the tightening member 32 are engaged with each other. As a result, the clip 2 is restricted to move toward the tip end side with respect to the holding tube 3, and the pair of arms 21 is locked in the closed state. Once the pair of arms 21 is locked in the closed state, the pair of arms 21 cannot return to the open state.

A tightening member 32 is provided in the inner region S of the holding tube 3, and a support surface 30s for supporting the tightening member 32 is integrally formed with the holding tube main body 30. The tightening member 32 is not separately from the holding tube main body 30. Therefore, when the engagement portion 24 is pulled to the base end side of the tightening member 32, the holding tube main body 30 is less likely to be broken. Further, the tightening member 32 hardly separates from the holding tube main body 30.

Since the tightening member 32 is formed in a ring shape, the tightening member 32 is reliably engaged with the engagement portion 24 and the pair of arms 21 is reliably locked in the closed state, even though a shape of the closed pair of arms 21 due to the shape of the tissue to be grasped is any shape.

Since the first support surface 301 is a surface substantially perpendicular to the central axis O1, when the engagement portion 24 is pulled to the base end side of the tightening member 32, the first support surface 301 is capable of reliably supporting the tightening member 32 so that the tightening member 32 not to move toward the base end side.

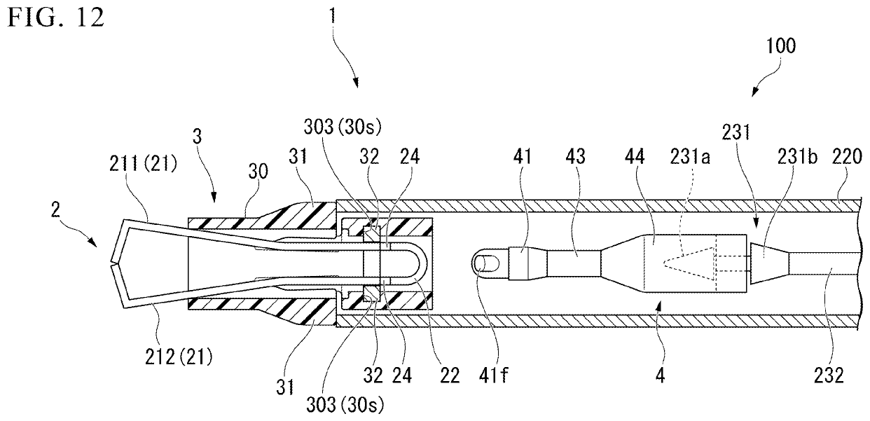

FIG. 12 is a figure showing the clip unit 1 that the clip 2 is separated.

The user further pulls the clip 2. When a tensile breaking force amount of, for example, 20N (Newton) to 90N is applied to the hook 41f, and the hook 41f breaks. The user retracts the sheath 220 and indwells the clip 2 that is in a state of ligating the tissue, in the body.

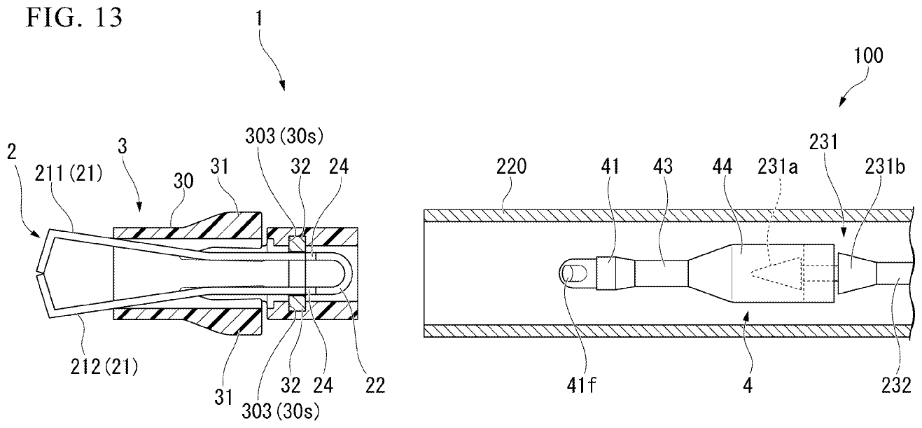

FIG. 13 is a figure showing the clip unit 1 after breaking.

When the hook 41f is broken, the energy of pulling the clip 2 toward the base end side by the connecting member 4 no longer acts on the clip 2, and a force (impact load) acts to move the clip 2 toward the tip side. Since the third support surface 303 supports the tightening member 32 from the tip side when an impact load is applied, the pair of arms 21 is reliably maintained in the closed state.

According to the clip device 100 and the clip unit 1 according to the embodiment, the ring-shaped tightening member 32 is provided in the inner region S of the holding tube 3, and the distance from the tissue grasping portion 23 that grasps the target object to the engagement portion that engages with the tightening member 32 is long. Therefore, for example, in a case that the shape of the object grasped by the tissue grasping portion 23 is large, the effect of the opening and closing operation of the tissue grasping portion 23 on the engagement portion 24 is small. As a result, the clip 2 that has the self-expanding force and is capable of re-grasping is securely locked in the closed state by the holding tube 3.

According to the clip device 100 and the clip unit 1 according to the embodiment, the holding tube 3 is made of an elastically deformable material such as resin in order to form the protrusion-retraction wing 31. Even in this case, the clip 2 is reliably locked in the closed state by the metal tightening member 32 provided in the inner region S of the holding tube 3.

As described above, the first embodiment of the present disclosure has been described in detail with reference to the drawings, but the specific configuration is not limited to the embodiment, and design changes and the like are included within the scope of the present disclosure. Also, the constituent elements shown in the above-described embodiment and modifications shown below may be combined as appropriate.

### Modified Example 1

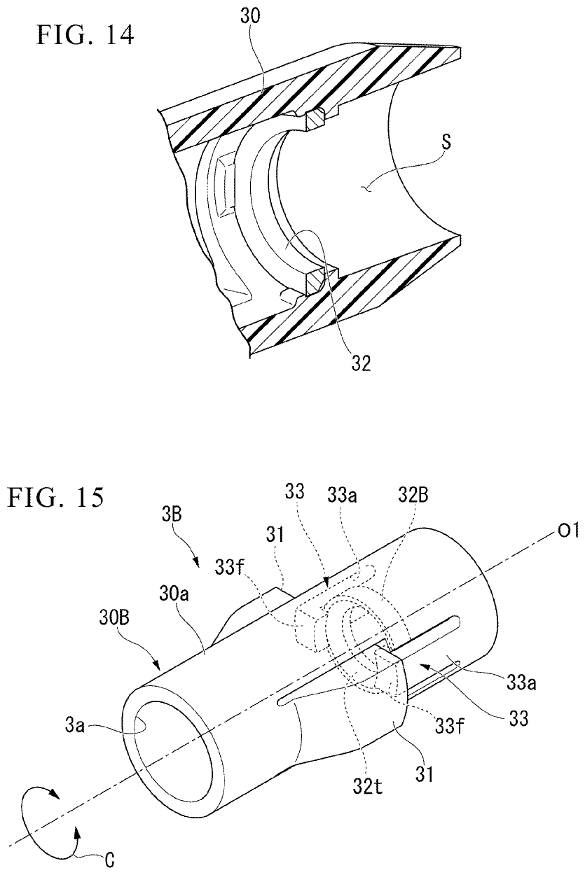

For example, in the above-described embodiment, the tightening member 32 is incorporated into the holding tube main body 30 by, for example, insert molding. However, the manner in which the tightening member 32 is incorporated is not limited to the embodiment. FIG. 14 is a perspective cross-sectional view showing different aspect of incorporation of the tightening member 32. The tightening member 32 may be pressed and fixed as shown in FIG. 14.

### Second Embodiment

A second embodiment of the present disclosure will be described with reference to FIGS. 15 to 19. In the following description, the same reference numerals are given to the same configurations as those already described, and redundant descriptions will be omitted. A clip unit 1B according to the second embodiment differs the holding tube 3 from the clip unit 1 according to the first embodiment.

FIG. 15 is a perspective view of the holding tube 3B.

A clip unit (endoscope treatment tool) 1B constitutes a clip device together with a clip introducer 200, such as the clip unit 1 of the first embodiment. The clip unit 1B includes a clip 2, a holding tube 3B having substantially cylindrical shape, and a connecting member 4. The holding tube 3B has a holding tube main body 30B formed in the cylindrical shape, protrusion-retraction wings 31, tightening member 32B, and a snap fit portion 33.

The holding tube main body 30B is made of a material softer than that of the clip 2, such as PPA (polyphthalamide), PA (polyamide), PEEK (polyetheretherketone), or other highly rigid resin material having appropriate elasticity. The holding tube main body 30B may be made of metal instead of a highly rigid resin material.

The protrusion-retraction wings 31 are a pair of projections projecting with respect to the outer peripheral surface 30a of the holding tube main body 30B, same as the first embodiment. The protrusion-retraction wings 31 are provided on both sides of the center axis O1.

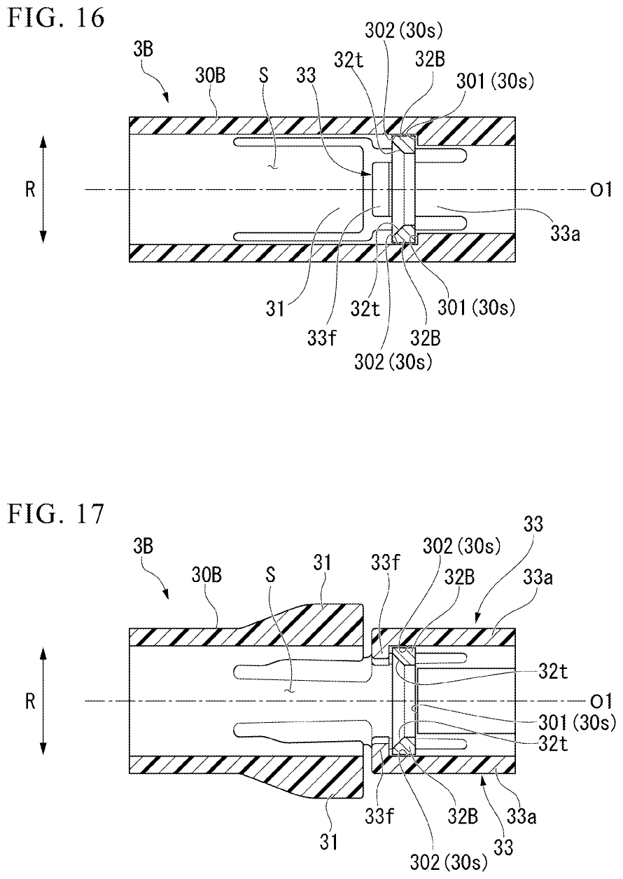

FIG. 16 is a cross-sectional view of the holding tube 3B along the central axis O1.

The tightening member 32B is a ring-shaped member provided in the inner region S of the holding tube 3B. The tightening member 32B is made of metal. A tapered surface 32t is formed on a tip end side of the tightening member 32B. The tapered surface 32t is formed such that an inner diameter of which gradually decreases from the tip end side toward the base end side. The tightening member 32B may be formed so as to be harder than the holding tube main body 30B, and may be formed of, for example, a highly rigid resin material instead of metal.

FIG. 17 is a cross-sectional view of the holding tube 3B along the central axis O1. The cross-sectional view shown in FIG. 17 is a cross-sectional view rotated 90 degrees about the central axis O1 with respect to FIG. 16.

The tightening member 32B is arranged such that the central axis of the tightening member 32B coincides with the central axis O1. The tightening member 32B is arranged on the base end side of the protrusion-retraction wing 31. The tightening member 32B is attached to the holding tube main body 30B by the snap fit portion 33. The base end side of the tightening member 32B is in contact with the support surface 30s formed on the holding tube main body 30B. The support surface 30s has a first support surface 301, and a second support surface 302.

The snap fit portion 33 is a pair of members provided on the pressing tube main body 30B. The snap fit portions 33 are provided on both sides of the center axis O1. The snap fit portions 33 have an elastic deformation portion 33a, and a hook 33f.

The elastically deforming portion 33a is fixed to the holding tube main body 30B only at the base end portion. The elastically deformable portion 33a elastically deforms as a cantilever with an end fixed to the holding tube main body 30B as a fixed end.

The hook 33f is provided at the end of the tip side of the elastically deformable portion 33a on, and is a convex portion protruding toward the inner region S side. The hook 33f fixes the tightening member 32B from the tip side.

As shown in FIG. 15, the snap-fit portion 33 is provided at a position overlapping the protrusion-retraction wing 31 in the circumferential direction C when viewed from the front in a direction horizontal to the center axis O1. From the viewpoint of the strength of the holding tube main body 30B, the snap fit portion 33 may be provided at a position that does not overlap with the protrusion-retraction wing 31 in the circumferential direction C in the front view.

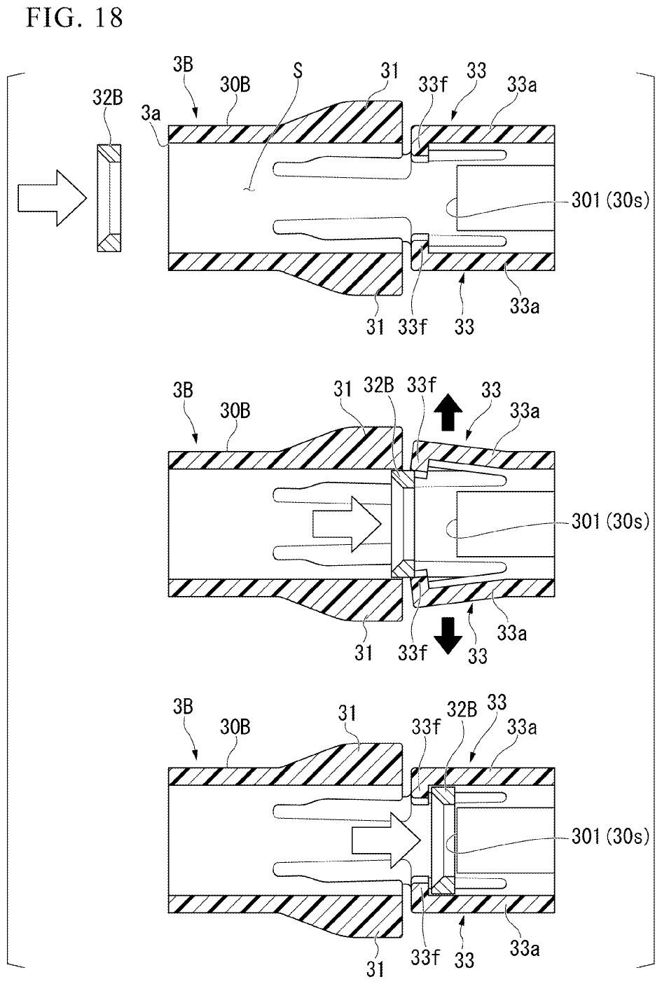

FIG. 18 is a figure illustrating a method of attaching the tightening member 32B to the holding tube main body 30B. The tightening member 32B is inserted into the internal space S from the tip opening 3a of the holding tube main body 30B. The fastening member 32B is inserted into the attachment position by pushing the hook 33f of the snap fit portion 33 outward in the radial direction R. When the tightening member 32B is inserted into an attached position, the hook 33f returns to its original position and fixes the tightening member 32B from the tip side.

According to the clip unit 1B according to the embodiment, since the ring-shaped tightening member 32B is provided in the inner region S of the holding tube 3B, the clip 2 having a self-expanding force and which is capable of being re-grasped is reliably locked in the closed state by the holding tube 3B.

According to the clip unit 1B according to the embodiment, the fastening member 32B is easily attached by the snap fit portion 33. Since the tightening member 32B is attached to the snap fit portion 33 from the tip end side, the tightening member 32B is not separated from the holding tube main body 30B when the engagement portion 24 is pulled to the base end side of the tightening member 32B.

According to the clip unit 1B according to the embodiment, the tightening member 32B has the tapered surface 32t. Therefore, when the engagement portion 24 is drawn to the base end side of the tightening member 32B, the engagement portion 24 easily passes through the tightening member 32B.

As described above, the second embodiment of the present disclosure has been described in detail with reference to the drawings, but the specific configuration is not limited to this embodiment, and design changes and the like are also included within the scope of the present disclosure. The constituent elements shown in the above-described embodiment and modified examples may be combined as appropriate.

### Modified Example 2

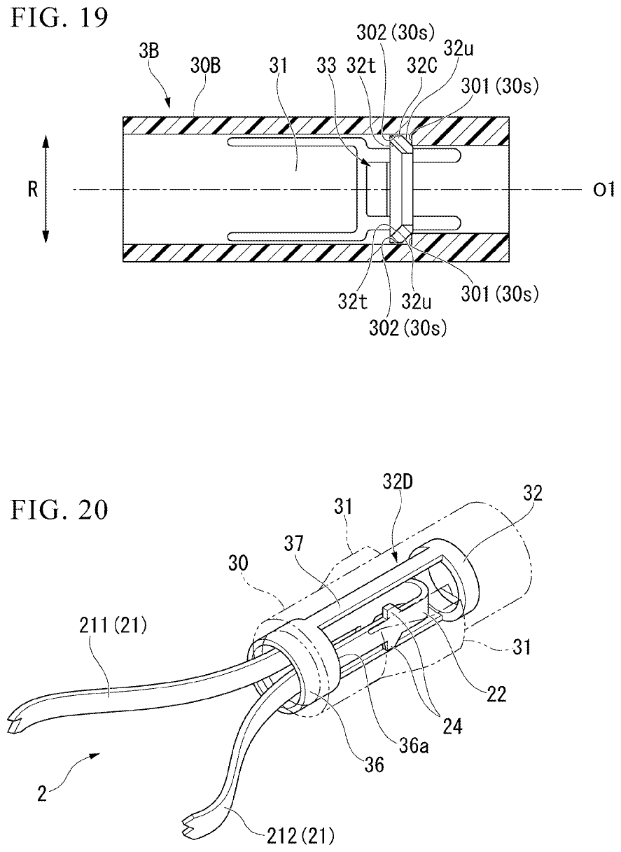

For example, in the above embodiment, the surfaces of the tightening member 32 and the tightening member 32B that contact with the first support surface 301 are substantially perpendicular to the central axis O1. However, the shapes of the tightening member 32 and the tightening member 32B are not limited to this. FIG. 19 is a cross-sectional view showing a tightening member 32C that is a modified example of the tightening member 32B. A second tapered surface 32u is formed on the base end side of the tightening member 32C. An outer diameter of the second tapered surface 32u gradually decreases from the tip end side toward the base end side. A tapered surface may be formed on the first support surface 301.

### Modified Example 3

For example, in the above embodiment, the tightening member 32 and the tightening member 32B are ring-shaped members. However, the shapes of the tightening member 32 and the tightening member 32B are not limited to this. FIG. 20 is a perspective view showing a tightening member 32D that is a modified example of the tightening member 32. The tightening member 32D is, for example, integrally formed of metal. The tightening member 32D has a tightening member 32, a holding pipe (supporting member) 36 having a cylindrical shape, and a connecting member 37 that connects the tightening member 32 with the holding pipe 36. A base end portion 36a of the holding pipe 36 is in contact with the holding pipe main body 30, and the holding pipe 36 is supported by the holding pipe main body 30 when a force is applied to the base end side. On the other hand, in the modified example, the tightening member 32 may not to be supported by the holding tube main body 30.

The holding pipe 36 protrudes from the holding tube main body 30 toward the tip side, and contacts with the pair of arms 21. The holding pipe 36 is made of a hard material such as metal, and the strength of the tip of the holding pipe 3 is enhanced.

### Modified Example 4

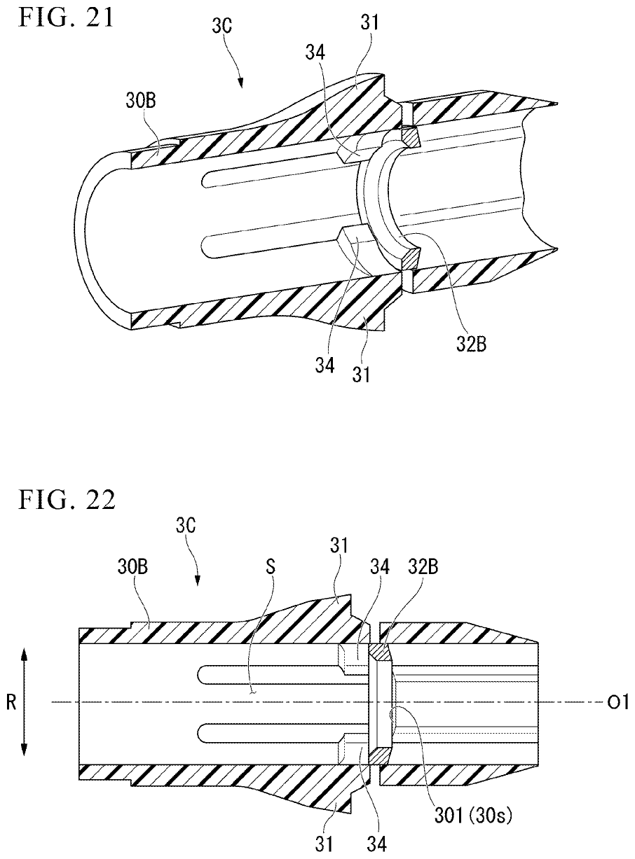

For example, in the above-described embodiment, the tightening member 32B is attached by the snap fit portion 33 provided on the holding tube 3B. However, the attachment manner of the tightening member 32B is not limited to this. FIGS. 21 and 22 are cross-sectional views showing a holding tube 3C that is a modified example of the holding tube 3B. The holding tube 3C has a holding tube main body 30B, a protrusion-retraction wing 31, a tightening member 32B, and a hook 34. The hooks 34 are protrusions projecting inward in the radial direction R from the inner peripheral surface of the protrusion-retraction wings 31. The hooks 34 are provided on both sides of the center axis O1. The tightening member 32B is inserted into the attachment position by pushing the protrusion-retraction wings 31 and the hooks 34 outward in the radial direction R. When the tightening member 32B is inserted into the attachment position, the hooks 34 return to its original position and fixe the tightening member 32B from the tip side. That is, the protrusion-retraction wings 31 and the hooks 34 function as the snap fit portion 33 of the above aspect.

### Modified Example 5

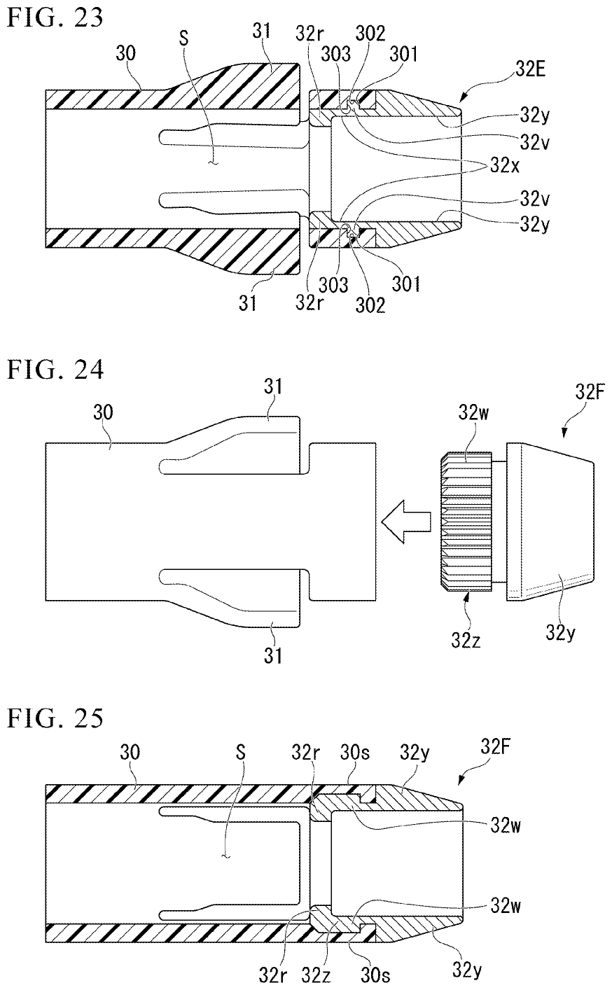

For example, in the above embodiment, the tightening member 32 is a ring-shaped member. However, the shape of the tightening member 32 is not limited to this. FIG. 23 is a cross-sectional view showing a tightening member 32E that is a modified example of the tightening member 32. The tightening member 32E is formed of metal and in a cylindrical shape. The tightening member 32E has a tip end portion 32x and a base end portion 32y. The tip end portion 32 x is inserted inside the holding tube main body 30. The base end portion 32y is exposed from the holding tube main body 30 toward the base end side, and has an outer peripheral surface that is continuous with the outer peripheral surface of the holding tube main body 30.

The tip end portion 32x has a ring portion 32r formed on the inner peripheral surface of the tip end portion 32x and a convex portion 32v formed on the outer peripheral surface of the tip end portion 32x. The ring portion 32r has a ring shape same as the tightening member 32, and engages with the engagement portion 24. The convex portion 32v is formed along the circumferential direction C, and contacts with the support surface 30s (the first support surface 301, the second support surface 302, and the third support surface 303).

In the tightening member 32E, since the tip end portion 32x and the base end portion 32y are integrally formed, the tightening member 32E is easy to handle. Therefore, the tightening member 32E is easily incorporated into the holding tube main body 30 by insert molding.

### Modified Example 6

FIG. 24 is a side view showing a tightening member 32F that is a modified example of the tightening member 32. FIG. 25 is a cross-sectional view showing the tightening member 32F. The fastening member 32F is formed of metal and in a cylindrical shape. The fastening member 32F has a tip end portion 32z and a base end portion 32y. The tip end portion 32z is inserted inside the holding tube main body 30.

The tip end portion 32z has a ring portion 32r formed on the inner peripheral surface of the tip end portion 32z and a convex portion 32w formed on the outer peripheral surface of the tip end portion 32z. The convex portion 32w is formed along the circumferential direction C, and contacts with the support surface 30s (the first support surface 301, the second support surface 302, and the third support surface 303). The outer peripheral surface of the protrusion 32w is serrated. Therefore, the tightening member 32F is easily incorporated into the holding tube main body 30 by press-fitting.

### Modified Example 7

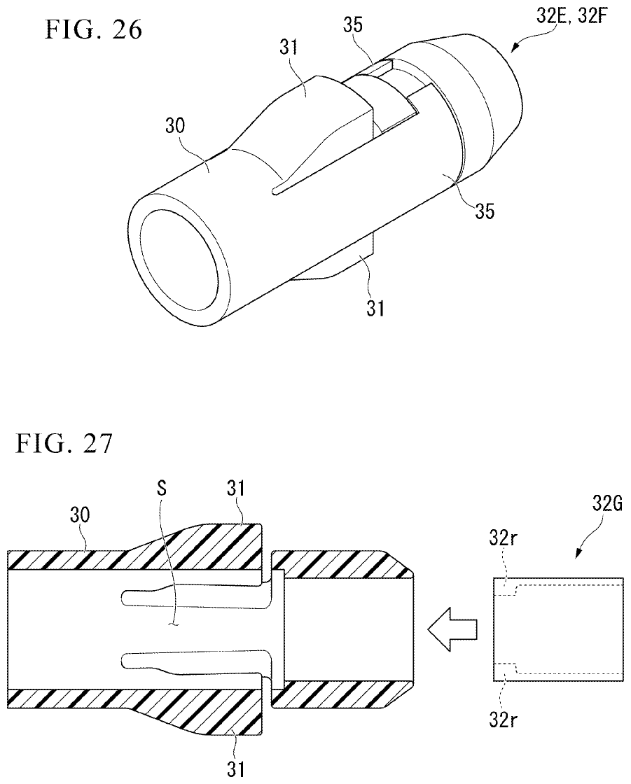

FIG. 26 is a figure showing another attachment aspect of the tightening member 32E and the tightening member 32F. The tightening member 32E and the tightening member 32F may be incorporated into the holding tube main body 30 by the snap fit portion 35 provided at the base end of the holding tube main body 30.

### Modified Example 8

FIG. 27 is a cross-sectional view showing a tightening member 32G that is a modified example of the tightening member 32. The tightening member 32G is made of metal and has a cylindrical shape. The entire tightening member 32G is inserted into the holding tube main body 30. A ring portion 32r is formed on the inner peripheral surface of the tightening member 32G. The tightening member 32G is easily incorporated into the holding tube main body 30 by press-fitting.

### Modified Example 9

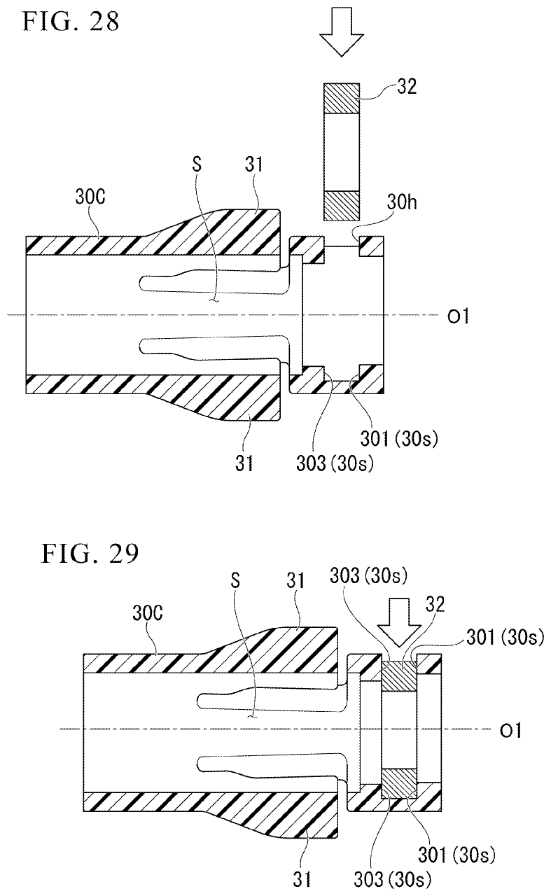

FIGS. 28 and 29 are cross-sectional views showing a holding tube main body 30C that is a modified example of the holding tube main body 30. The holding tube main body 30C has a through hole 30h through which the tightening member 32 is capable of being inserted into the internal space S from a direction perpendicular to the central axis O1. As shown in FIG. 29, the tightening member 32 inserted into the internal space S is supported by the first support surface 301 and the third support surface 303.

### Modified Example 10

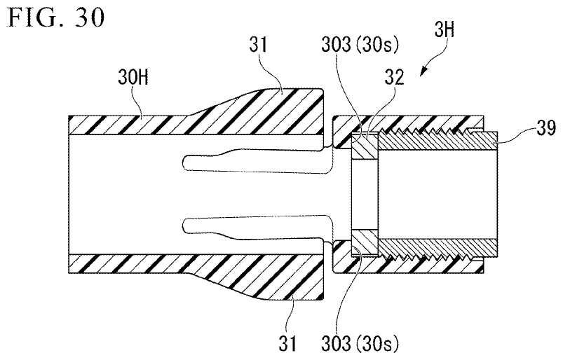

FIG. 30 is a cross sectional view showing a holding tube 3H that is a modified example of the holding tube 3. The holding tube 3H has a holding tube body 30H formed in a cylindrical shape, protrusion-retraction wings 31, a tightening member 32, and a fixing member 39. The holding tube main body 30H is the same as the holding tube main body 30 of the first embodiment except that the first support surface 301 is not provided.

The fixing member 39 is formed of metal, and in a cylindrical shape. The fixing member 39 is inserted into the internal space S of the holding tube main body 30H, and fixes the tightening member 32 from the base end side. The holding tube main body 30H and the fixing member 39 are fixed by, for example, a screw or a barb fitting.

### Third Embodiment

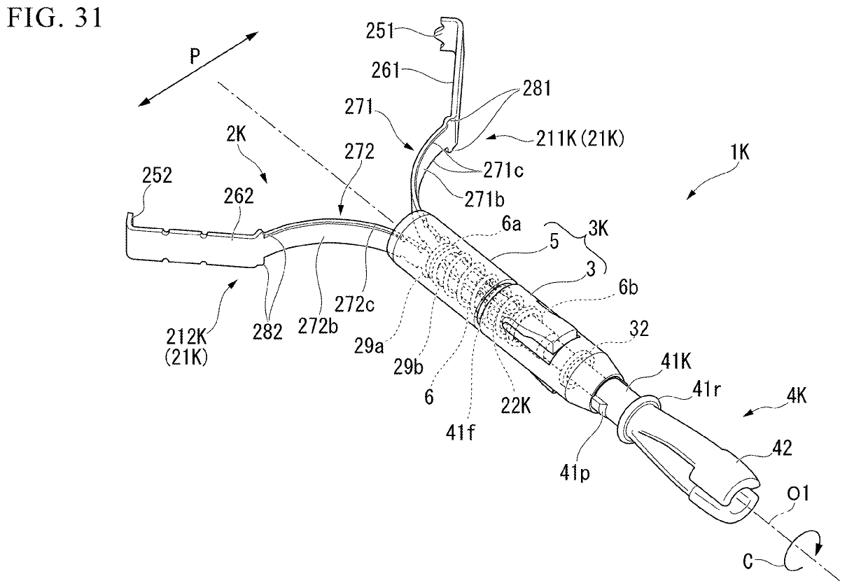

A third embodiment of the present disclosure will be described with reference to FIGS. 31 to 40. In the following description, the same reference numerals are given to the same configurations as those already described, and redundant descriptions will be omitted.

### [Clip Unit 1K]

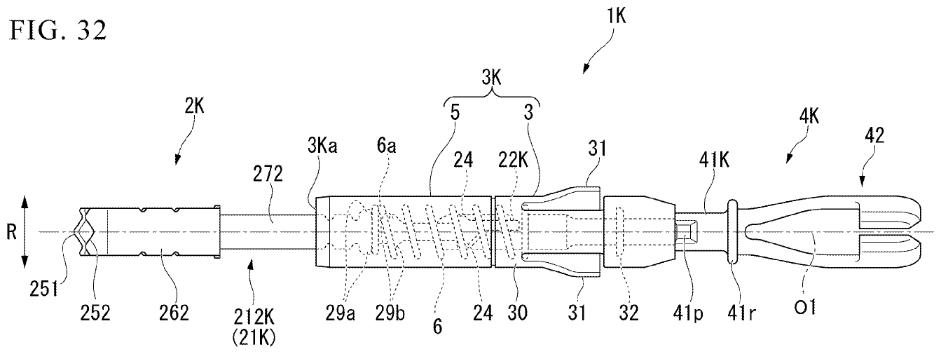

FIG. 31 is a perspective view of the clip unit 1K according to the embodiment. FIG. 32 is a side view of the clip unit 1K. A clip unit (endoscopic treatment tool) 1K is used by loading it into a clip introducer 200, like the clip unit 1 of the first embodiment. The clip unit 1K includes a clip 2K, a holding tube 3K, a connecting member 4K, and an elastic member 6.

The clip 2K is formed by bending a metal plate at a center portion, such as the clip 2 of the first embodiment. The clip 2K has a pair of arms 21K that is openable and closeable, and a connecting portion 22K that connects with the pair of arms 21K.

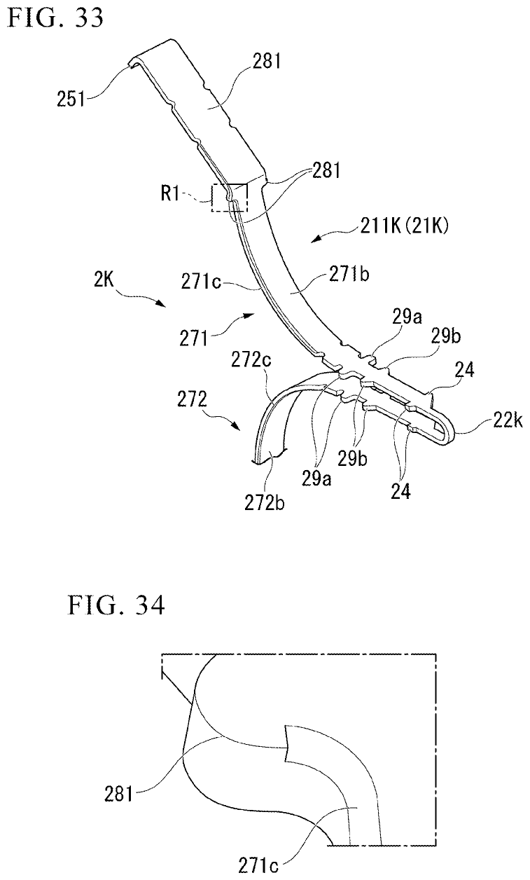

FIG. 33 is a perspective view of a pair of arms 21K.

The pair of arms 21K has a first arm 211K and a second arm 212K. The first arm 211K and the second arm 212K are arranged symmetrically with respect to the central axis O1 in the longitudinal direction of the clip unit 1.

The first arm 211K has a tissue grasping portion 251, a flat grasping portion 261 having a plate-like shape, and a sliding portion 271 from the tip end side to the base end side. The second arm 212K has a tissue grasping portion 252, a flat plate-like grasping portion 262, and a sliding portion 272 from the tip end side to the base end side.

The tissue grasping portion 251 is formed by bending the tip end of the first arm 211K inward. The tissue grasping portion 252 is formed by bending the tip end of the second arm 212K inward. As shown in FIG. 32, the tissue grasping portion 251 and the tissue grasping portion 252 are formed in an asymmetrical shape with respect to the central axis O1. Therefore, when attaching the pair of arms 21K to the connecting member 4K, the user easily recognizes the posture of attaching the pair of arms 21K to the connecting member 4K.

FIG. 34 is an enlarged view of a region R1 shown in FIG. 33.

The sliding portions 271 and 272 are portions that are elastically deformed when the pair of arms 21K are drawn into the holding tube 3K. The sliding portions 271 and 272 have chamfered portions 271c and 272c in which ends on outer peripheral surfaces 271b and 272b on the outer side in the opening/closing direction P are chamfered along the longitudinal direction. Since the sliding portions 271 and 272 have the chamfered portions 271c and 272c, when the pair of arms 21K are drawn into the holding tube 3K, the sliding portions 271 and 272 are less likely to get caught in the tip opening 3Ka of the holding tube 3K.

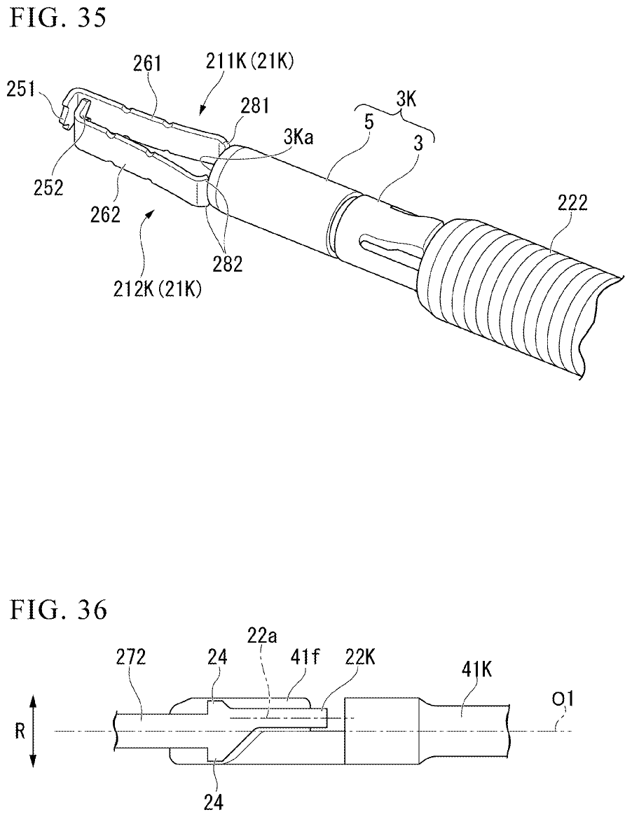

FIG. 35 is a figure showing a pair of arms 21K partly accommodated in the holding tube 3K.

A stopper 281 is formed in a direction orthogonal to the opening/closing direction P at the connecting portion between the grip portion 261 and the sliding portion 271. A stopper 282 is formed in a direction orthogonal to the opening/closing direction P at the connecting portion between the grip portion 262 and the sliding portion 272. As shown in FIG. 35, when the pair of arms 21K is drawn into the holding tube 3K, the stoppers 281 and 282 engage with the tip opening 3Ka of the holding tube 3K. That is, the stoppers 281 and 282 restrict the position at which the pair of arms 21K is drawn into the holding tube 3K.

As shown in FIG. 32, the sliding portions 271 and 272 are provided with a first support portion 29a that supports the tip end side of the tip end portion 6a of the elastic member 6, and a second support portion 29b that supports the base end side of the tip end portion 6a. The first support portion 29a and the second support portion 29b sandwich the tip end portion 6a of the elastic member 6 therebetween. Therefore, the expansion/contraction action of the elastic member 6 with respect to the pair of arms 21K is stabilized.

FIG. 36 is a side view of the base ends of the pair of arms 21K.

Engagement portions 24 projecting in a direction perpendicular to the central axis O1 are formed on the base end side of the first arm 211K and the base end side of the second arm 212K. The tip end side of the engagement portions 24 are formed to have an acute slope, and the connecting portion 22 side of the engagement portions 24 are formed to have an obtuse slope. The engagement portions 24 are provided in an accommodated region T that is accommodated in the inner region S of the holding tube 3K. The accommodated region T is a region that is always accommodated in the inner region S of the holding tube 3K when the pair of arms 21K are opened and closed.

The first arm 211K and the second arm 212K are connected by a connecting portion 22K on the base end side, and are provided so as to be openable and closable toward the tip end side. The connecting portion 22K is formed to be bent to have a U-shape and is connected to the connecting member 4K.

As shown in FIG. 36, an axis 22a along the longitudinal axis of the connecting portion 22K is located outside in the radial direction R from the center axis O1. Therefore, the connecting member 22K is easy to hook on the hook 41f formed in the shape of a hook.

The holding tube 3K has a holding pipe 5 and a holding tube 3. The holding pipe 5 is a metal cylindrical member. The holding pipe 5 is press-fitted into the tip end of the holding tube 3. The holding tube 3K and the holding pipe 5 may be connected by heat welding, adhesion, or screwing.

A tip end opening 3Ka formed on the tip end side of the holding pipe 5 engages with the sliding portions 271 and 272 of the pair of arms 21K. In the tip end opening 3Ka, burr is removed by surface treatment, for example, magnetic barrel treatment or the like, and is less likely to be caught by the sliding portions 271 and 272. The tip end opening 3Ka may be treated to a surface treatment, for example, nitriding or the like, to increase the surface hardness. By increasing the surface hardness of the tip end opening 3Ka, the tip end opening 3Ka is less likely to be caught by the sliding portions 271 and 272.

The connecting member 4K is connected with the connecting portion 22K of the clip 2K. The connecting member 4K is connected with the arrowhead hook portion 231 passing through the sheath 220. The connecting member 4K includes an inserting portion 41K inserted into the inner space of the holding tube 3K, and a connecting portion 42 provided at the base end of the inserting portion 41K.

The insertion portion 41K has a hook 41f, a marking 41p, and a stopper 41r at a tip end portion of the insertion portion 41K. The marking 41p is a convex portion that protrudes outward in the radial direction R, and is formed only partially in the circumferential direction C of the insertion portion 41K. Therefore, when attaching the pair of arms 21K to the connecting member 4K, the user easily recognizes the posture of attaching the pair of arms 21K to the connecting member 4K.

The stopper 41r is a convex portion that protrudes outward in the radial direction R, and is formed in a ring shape along the circumferential direction C. The stopper 41r engages with the base end of the holding tube 3K. That is, the stopper 41r restricts the position where the connecting member 4K is drawn into the holding tube 3K.

The elastic member 6 is a spring that biases the clip 2K to the tip end side. As shown in FIG. 31, an end turn is formed on a base end portion 6b of the elastic member 6, thereby the elastic member 6 is stably engaged with the connecting member 4K.

### [Actions and Effects of the Clip Unit 1K]

Next, actions and effects of the clip unit 1K will be described.

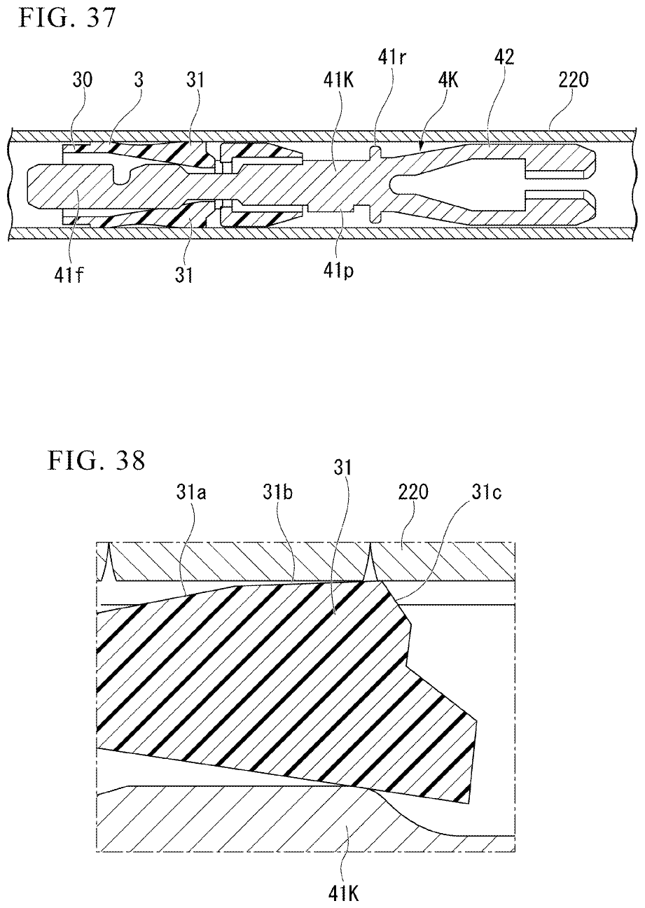

FIG. 37 is a cross-sectional view showing the clip unit 1K loaded into the sheath 220 of the clip introducer 200. In FIG. 37, description of the holding pipe 5, the elastic member 6, the arrowhead hook portion 231 connected with the connecting member 4K, and the like are omitted.

FIG. 38 is a cross-sectional view of the protrusion-retraction wing 31.

The protrusion-retraction wing 31 is in the retracted state by being pressed by the inner peripheral surface of the sheath 220. In the protrusion-retraction wing 31, a first slope 31a, a second slope 32b, and a third slope 31c are provided from the tip end side to the base end side. The first slope 31a, the second slope 32b, and the third slope 31c have different normal directions with respect to the central axis O1. Therefore, the protrusion-retraction wing 31 is hardly caught on the sheath 220.

As shown in FIG. 37, the inner peripheral surface of the protrusion-retraction wing 31 sandwiches the insertion portion 41K of the connecting member 4K. The protrusion-retraction wing 31 restricts a movement of the connecting member 4K from the tip end side toward the base end side. As a result, a positional displacement of the holding tube 3K and the connecting member 4K is relatively suppressed.

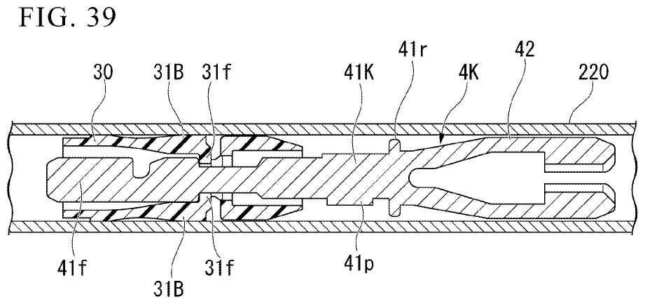

FIG. 39 is a cross-sectional view showing protrusion-retraction wings 31B that are a modified example of the protrusion-retraction wing 31.

The protrusion-retraction wings 31B have a hook 31f protruding inward in the radial direction R on the base end side of the inner peripheral surface. The protrusion-retraction wings 31B are more reliably restrict the movement of the connecting member 4K from the tip end side toward the base end side.

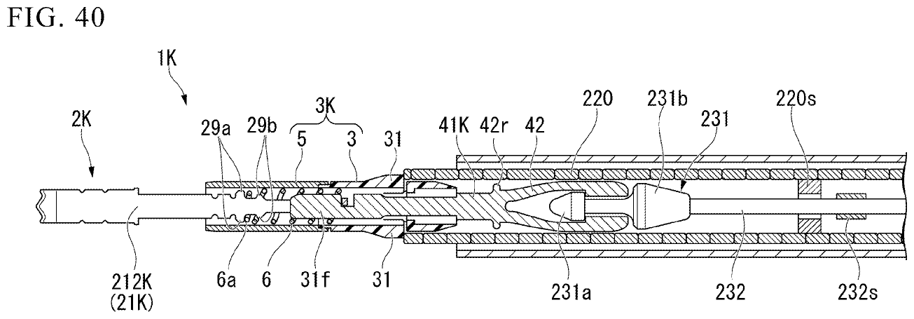

FIG. 40 is a side view showing the clip unit 1K projecting from the sheath 220 toward the base end. The user advances the arrowhead hook portion 231 by advancing the slider 242 along the operating section main body 241. The user advances the clip unit 1K until the protrusion-retraction wings 31 expose from the sheath 220. When the tip end side of the protrusion-retraction wings 31 expose from the sheath 220, the protrusion-retraction wings 31 return to the projected state, which is the basic posture from the retracted state.

A diameter-reducing portion 220s having an inner diameter smaller than that of other portions is provided on the inner peripheral surface of the sheath 220 on the tip end side. An enlarged diameter portion 232s having a larger outer diameter than other portions is provided on the tip end side of the wire 232. The enlarged diameter portion 232s is capable of passing through the diameter-reducing portion 220s, but the enlarged diameter portion 232s contacts with the inner peripheral surface of the diameter-reducing portion 220s during passing. Therefore, the advancing/retreating operation of the slider 242 for making the enlarged diameter portion 232s to pass through the diameter-reducing portion 220s requires a larger force than a normal advance/retreat operation. The user recognizes that the enlarged diameter portion 232s is passing through the diameter-reducing portion 220s based on a feeling that a load is higher than usual when the slider 242 is advanced and retracted. By providing the reduced-diameter portion 220s and the enlarged diameter portion 232s at appropriate positions, it is possible to prevent the user from moving the wire 232 to an unintended area.

According to the clip unit 1K according to the embodiment, since the ring-shaped tightening member 32 is provided in the inner region S of the holding tube 3K, the clip 2K that is capable of being re-grasped is securely locked in the closed state by the holding tube 3K.

The third embodiment of the present disclosure has been described in detail with reference to the drawings, but the specific configuration is not limited to the embodiment, and design changes and the like are also included within the scope of the present disclosure. The constituent elements shown in the above-described embodiment and modifications can be combined as appropriate.

### Modified Example 11

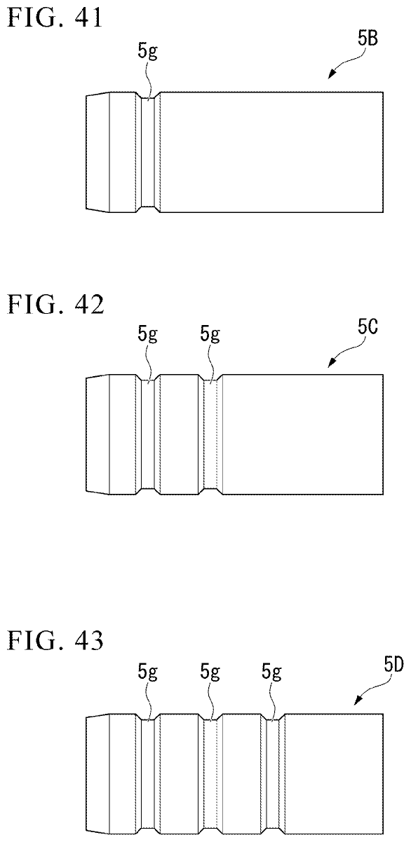

FIGS. 41 to 43 are figures showing a holding pipe 5B, a holding pipe 5C, and a holding pipe 5D, which are modified examples of the holding pipe 5. The holding pipe 5B, the holding pipe 5C, and the holding pipe 5D have grooves 5g extending in the circumferential direction C on their outer peripheral surfaces. The holding pipe 5B, the holding pipe 5C, and the holding pipe 5D have different numbers of grooves 5g. The user easily recognizes the type of the clip unit 1K by using different holding pipes with different appearances for each type of the clip unit 1K.

### Engagement Portion

What is claimed is:

1. An endoscopic treatment tool comprising:
a clip having a plurality of arms freely open and close at a distal side of the arm;
a holding tube into which at least a portion on a proximal side is inserted, and
a connecting member connecting with the clip;
wherein the holding tube includes a tightening member provided in an inner region of the holding tube, and a protrusion-retraction wing configured to protrude and retract with respect to an outside in a radial direction,
the tightening member is provided on a proximal side from the protrusion-retraction wing,
the protrusion-retraction wing is configured to transfer to a protruded state protruding outside in the radial direction from an outer peripheral surface of the holding tube and a retracted state retracting inward the holding tube in the radial direction,
when the protrusion-retraction wing is in the retracted state, the protrusion-retraction wing engages with the connecting member so that the clip is restricted to move toward the proximal side with respect to the holding tube,
the clip has an engagement portion on an accommodation region accommodated in the inner region, and
a movement of the clip toward a distal side with respect to the holding tube is restricted by engaging the engagement portion moved to the proximal side with the tightening member.

2. The endoscopic treatment tool according to claim 1,
wherein the tightening member is formed in a ring shape.

3. The endoscopic treatment tool according to claim 1,
wherein the tightening member is made of a material harder than an other portion of the holding tube.

4. The endoscopic treatment tool according to claim 3,
wherein the tightening member is made of a metal, and
the other portion of the holding tube is made of resin.

5. The endoscopic treatment tool according to claim 1,
Wherein the holding tube has a support surface supporting the tightening member from the proximal side.

6. The endoscopic treatment tool according to claim 5,
wherein the holding tube has a support surface supporting the tightening member from the distal side.

7. The endoscopic treatment tool according to claim 1,
wherein the tightening member is attached to the holding tube by a snap fit portion provided on the holding tube.

8. The endoscopic treatment tool according to claim 1,
wherein the tightening member is attached to the holding tube by an insert molding.

9. The endoscopic treatment tool according to claim 1,
wherein the tightening member is supported by connecting a support member, and
the holding tube supports the support member from the proximal side.

10. The endoscopic treatment tool according to claim 1,
wherein the tightening member has a distal end portion inserted into the holding tube, and a proximal end portion exposed from the holding tube toward the proximal side, and
a ring portion engaged with the engagement portion is formed on an inner peripheral surface of the distal end portion.

11. The endoscopic treatment tool according to claim 1,
wherein the protrusion-retraction wing includes a hook on an inner peripheral surface of the protrusion-retraction wing, and the hook protrudes inward in the radial direction, and
the hook engages with the connecting member when the protrusion-retraction wing is in the retracted state.

12. The endoscopic treatment tool according to claim 1,
wherein the arm includes a stopper elongated in a direction crossing to an open-close direction of the arm, and
a movement of the clip toward the proximal side with respect to the holding tube is restricted by engaging the stopper with an opening at a distal end side of the holding tube.

13. The endoscopic treatment tool according to claim 1,
wherein the connecting member includes an insertion portion inserted into the inner region of the holding tube, and a connecting portion provided at a proximal end side of the insertion portion,
the connecting member has a width smaller than an inner diameter of the holding tube and larger than an inner diameter of the tightening member in the radial direction, and
a movement of the tightening member toward the distal side with respect to the tightening member is restricted by contacting the connecting member to the tightening member.

14. A clip device comprising:
an endoscopic treatment tool, and a clip introducer,
wherein the clip introducer includes a sheath, and a wire inserted thorough the sheath,
the endoscopic treatment tool includes:
a clip having a plurality of arms in which distal end side portions freely open and close;
a holding tube having a cylindrical shape and into which at least a part of a proximal end side of the clip is inserted; and
a connecting member connecting the clip with the wire,
the holding tube includes a tightening member provided in an inner region of the holding tube, and a protrusion-retraction wing configured to protrude and retract in a radial direction,
the tightening member is provided at a proximal side from the protrusion-retraction wing,
the protrusion-retraction wing is configured to transfer to a protruded state protruding outside in the radial direction from an outer peripheral surface of the holding tube and a retracted state retracting inward the holding tube in the radial direction,
when the protrusion-retraction wing is in the retracted state, the protrusion-retraction wing engages with the connecting member so that the clip is restricted to move toward the proximal side with respect to the holding tube,
the clip has an engagement portion on an accommodation region accommodated in the inner region, and
a movement of the clip toward a distal side with respect to the holding tube is restricted by engaging the engagement portion moved to the proximal side with the tightening member.

15. The clip device according to claim 14,
wherein the tightening member is arranged inside the sheath when the protrusion-retraction member is in the protruded state.
